(19) United States
(12) Patent Application Publication
(10) Pub. No.: US 2021/0275169 A1
(43) Pub. Date: Sep. 9, 2021

(54) Surgical Instrument And Linear Stapler

(71) Applicant: Suzhou Intocare Medical Technology Co., Ltd, Suzhou, Jiangsu (CN)

(72) Inventors: Ronghua Xu, Suzhou, Jiangsu (CN); Hui Zhang, Suzhou, Jiangsu (CN); Yunfeng Du, Suzhou, Jiangsu (CN); Dianchen Liu, Suzhou, Jiangsu (CN)

(21) Appl. No.: 17/273,062

(22) PCT Filed: Nov. 14, 2019
(86) PCT No.: PCT/CN2019/118394

§ 371 Date: Mar. 3, 2021

(30) Foreign Application Priority Data
Jan. 24, 2019 (CN) 201910067871.8

(51) Int. Cl. A61B 17/068; A61B 17/072; A61B 17/04; A61B 90/00

(52) U.S. Cl. CPC A61B 17/0686; A61B 17/072; A61B 17/0482; A61B 2090/0811; A61B 2017/07257; A61B 2017/07271; A61B 90/08

(57) ABSTRACT

A surgical instrument and a linear surgical stapler are provided. The instrument includes: a transmission assembly provided with a mounting part for mounting a suturing mechanism, in which the suturing mechanism includes an anvil assembly and a staple cartridge assembly, the staple cartridge assembly includes a staple cartridge for accommodating staples, the transmission assembly is connected to the anvil assembly and drives the anvil assembly to move relative to the staple cartridge assembly; a staple pushing assembly configured for pushing the staples to move toward the anvil assembly, and comprising a first threaded rod and a firing nut sleeved on the first threaded rod; and a drive assembly, connected to the transmission assembly to drive the transmission assembly to move; a firing assembly connected to the first threaded rod and driving the first threaded rod to rotate, so that the firing nut moves relative to the first threaded rod.

The present application claims priority of the Chinese Patent Application No. 201910067871.8 filed on Jan. 24, 2019, the disclosure of which is incorporated herein by its reference in its entirety as part of the present application.

### TECHNICAL FIELD

The present disclosure relates to a surgical instrument and a linear surgical stapler, and belongs to the field of medical instruments.

### BACKGROUND

A linear surgical stapler, also known as a digestive tract closer, is an instrument that insert a plurality of staples staggered each other into a target tissue in a straight line. The linear surgical stapler is one of the instruments widely used to replace a linearly manual suturing, is mainly used to suture and close the stumps of stomach, duodenum, small intestine, and colon and is a full-layer eversion suturing instrument.

A manner of inserting the staples into the target tissue by the linear surgical stapler in the prior art is usually that a transmission device pushes an anvil to push out and fire all of the staples at one time. In this case, the staples are prone to having large and small heads, a dovetail and the like so that an inserting effect is poor, resulting in a poor suturing effect and waste of staples. In addition, because a large number of staples are fired at one time, a part of the staples cannot be firmly inserted into the target tissue, resulting in tissue leakage. In addition, because a large number of staples need to be fired at one time, a relatively large firing force is needed, which causes the anvil with a cantilever beam structure to deform after a plurality of firings.

### SUMMARY

The present disclosure aims to provide a surgical instrument and a linear surgical stapler, which fire the staples sequentially, and thus can reduce the firing force to avoid the deformation of the anvil, and have a better insertion effect of the staples into the tissue, a better suturing effect and surgical safety.

In order to achieve the above objects, the technical solutions of the present disclosure are provided as follows. A surgical instrument comprises: a transmission assembly, in which the transmission assembly is provided with a mounting part for mounting a suturing mechanism, the suturing mechanism comprises an anvil assembly and a staple cartridge assembly, the staple cartridge assembly comprises a staple cartridge for accommodating staples, the anvil assembly comprises an anvil matched with the staple cartridge, and the transmission assembly is connected to the anvil assembly and drives the anvil assembly to move relative to the staple cartridge assembly; a staple pushing assembly configured for pushing the staples to move toward the anvil assembly, in which the staple pushing assembly comprises a first threaded rod provided on the staple cartridge assembly and a firing nut sleeved on the first threaded rod, an axis direction of the first threaded rod and the firing nut is defined as a first axis direction; and a drive assembly, connected to the transmission assembly to drive the transmission assembly to move. The surgical instrument further comprises a firing assembly provided on the anvil assembly, the firing assembly is connected to the first threaded rod and drives the first threaded rod to rotate, so that the firing nut moves relative to the first threaded rod in the first axis direction.

For example, the firing assembly comprises a firing motor, a firing input shaft provided on the firing motor, and a direction-changing transmission device respectively connected to the firing input shaft and the first threaded rod; and if the suturing mechanism is closed, the firing motor drives the first threaded rod to rotate by the direction-changing transmission device.

For example, the direction-changing transmission device comprises a first bevel gear sleeved on the firing input shaft and a second bevel gear sleeved on the first threaded rod, and the first bevel gear and the second bevel gear are engaged with each other.

For example, the transmission assembly comprises a transmission rod, the transmission rod is connected to the drive assembly and rotates under an action of the drive assembly, an axis direction of the transmission rod is defined as a second axis direction, and the first axis direction is perpendicular to the second axis direction.

For example, the transmission rod is a second threaded rod, and a threaded sleeve is sleeved on the second threaded rod; the drive assembly drives the second threaded rod to rotate, so that the threaded sleeve moves relative to the second threaded rod in the second axis direction.

For example, the transmission assembly further comprises a closure assembly sleeved on the second threaded rod, the closure assembly comprises a first closure rod and a second closure rod, the threaded sleeve is fixedly connected to the first closure rod and the second closure rod, the threaded sleeve is provided between the first closure rod and the second closure rod; the first closure rod is connected to the anvil assembly, the second closure rod is provided closer to the drive assembly than the first closure rod; if the drive assembly drives the second threaded rod to rotate, the threaded sleeve moves relative to the second threaded rod in the second axis direction, thereby driving the first closure rod and the second closure rod to move in the second axis direction.

For example, the surgical instrument further comprises a safety switch serving as a stopper, the safety switch is provided on the closure assembly, the safety switch comprises a switching knob and a switching shaft, the switching shaft is connected to the switching knob, and the second closure rod is provided with a through hole adapted to the switching shaft; and if the suturing mechanism is closed, the switching knob is toggled, the switching shaft is inserted into the through hole to lock the closure assembly.

For example, the anvil assembly further comprises an upper jaw arm and a lower jaw arm, the upper jaw arm is provided at an outer side of the staple cartridge, the upper jaw arm is provided with a slot hole for accommodating the first threaded rod and the firing nut, a first bearing for fixing the first threaded rod is provided in the slot hole; the lower jaw arm is provided with a hollow chamber for accommodating the firing assembly, and the firing assembly is mechanically fixed in the hollow chamber.

For example, the surgical instrument further comprises a handle, the transmission rod is fixed to the handle by a second bearing, the drive assembly is provided in the handle, and the drive assembly comprises a drive motor, an input shaft connected to the drive motor, and a transmission member respectively connected to the input shaft and the transmission rod.

For example, the surgical instrument further comprises an induction switch for sensing a position of the staple cartridge assembly, and the induction switch is provided in the handle.

The embodiments of the disclosure further provides a linear surgical stapler, comprising a surgical instrument as described above and the suturing mechanism, in which the suturing mechanism is mounted on the mounting part.

Compared with the prior art, the beneficial effects of the embodiments of the present disclosure at least are as follows. The surgical instrument and the linear surgical stapler of the present disclosure are provided with the staple pushing assembly and the firing assembly. If the suturing mechanism is closed, the firing assembly drives the first threaded rod of the staple pushing assembly to rotate so as to drive the firing nut sleeved on the first threaded rod to move relative to the first threaded rod in the first axis direction, so that the staples in the staple cartridge are fired sequentially. Therefore, there is no problem such as large and small heads, dovetail and the like. In addition, the surgical instrument and the linear surgical stapler fire the staples sequentially, which can reduce the firing force to avoid the deformation of the anvil, and have a better insertion effect of the staples into the tissue, a better suturing effect and surgical safety.

The above description is only an outline of the technical solutions of the embodiments of the present disclosure. In order to better understand the technical solutions of the embodiments of the present disclosure so that they can be implemented according to the contents of the specification, the following detailed description will be made with the exemplary embodiments of the present disclosure in conjunction with the accompanying drawings.

### BRIEF DESCRIPTION OF THE DRAWINGS

FIG. 1 is a schematic diagram of an external structure of a surgical instrument according to embodiments of the present disclosure;

FIG. 2 is a schematic diagram of an overall structure of the surgical instrument according to the embodiments of the present disclosure;

FIG. 3 is a schematic diagram of a distal end of the surgical instrument according to the embodiments of the present disclosure;

FIG. 4 is a schematic structural diagram of a transmission assembly of the surgical instrument according to the embodiments of the present disclosure;

FIG. 5 is a schematic structural diagram of a proximal end of the surgical instrument according to the embodiments of the present disclosure;

FIG. 6 is a schematic structural diagram of a linear stapler in an initial state according to the embodiments of the present disclosure;

FIG. 7 is a schematic structural diagram of the linear stapler in a closed state according to the embodiments of the present disclosure;

FIG. 8 is a schematic structural diagram of the linear stapler in a firing state according to the embodiments of the present disclosure.

### DETAILED DESCRIPTION

The exemplary specific implemented modes of the present disclosure will be described in further detail below in conjunction with the drawings and embodiments. The following embodiments are intended to illustrate the present disclosure, but not to limit the scope of the present disclosure.

In the description of the present disclosure, it should be noted that the orientation or positional relationship indicated by the terms such as “center”, “upper”, “lower”, “left”, “right”, “front”, “rear”, “axis direction”, “inner”, and “outer” are based on the orientation or positional relationship shown in the drawings, which are merely intended for describing the present disclosure and simplifying the description, but do not indicate or imply that the concerned device or element shall have a specific orientation, or be constructed and operated in a specific orientation, so that these terms cannot be understood as a limitation to the present disclosure. In addition, the terms such as “first”, “second”, and “third” are merely used for descriptive purposes, and cannot be understood as indicating or implying relative importance.

In the description of the present disclosure, it should be noted that the terms such as “mounting”, “connecting” and “connection” should be understood in a general sense, unless otherwise clearly specified and limited. For example, these terms may refer to a fixed connection, a detachable connection or an integral connection, a mechanical connection or an electrical connection, a direct connection or an indirect connection through an intermediate medium, or an internal communication between two elements. For a person of ordinary skill in the art, the specific meanings of the foregoing terms in the present disclosure may be understood according to specific situations.

In addition, the technical features involved in different embodiments of the present disclosure described below may be combined with each other as long as there is no conflict therebetween.

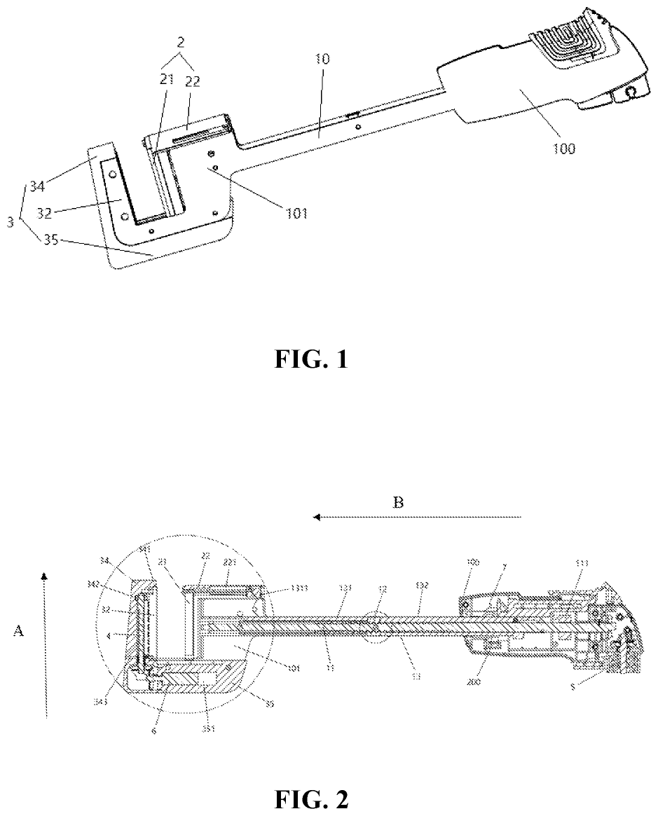

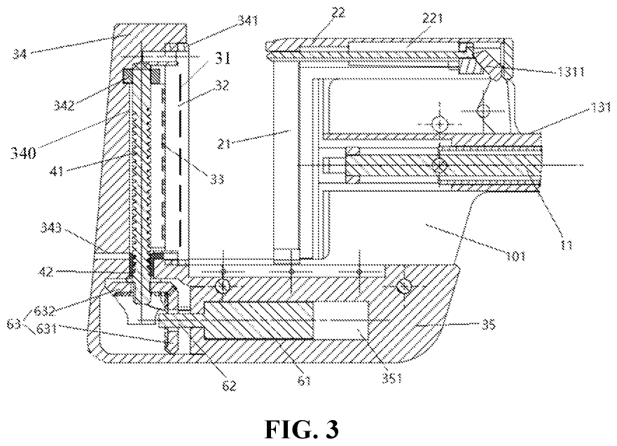

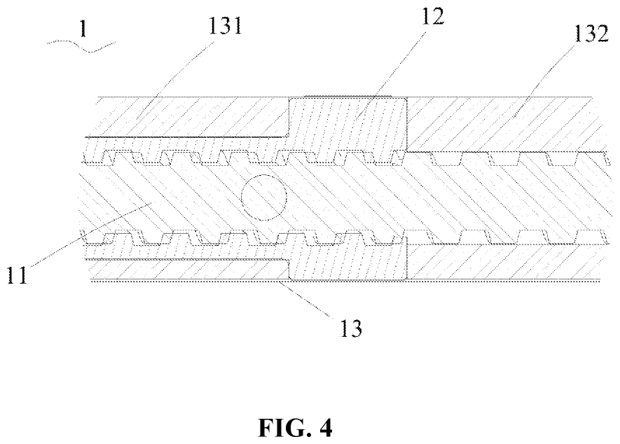

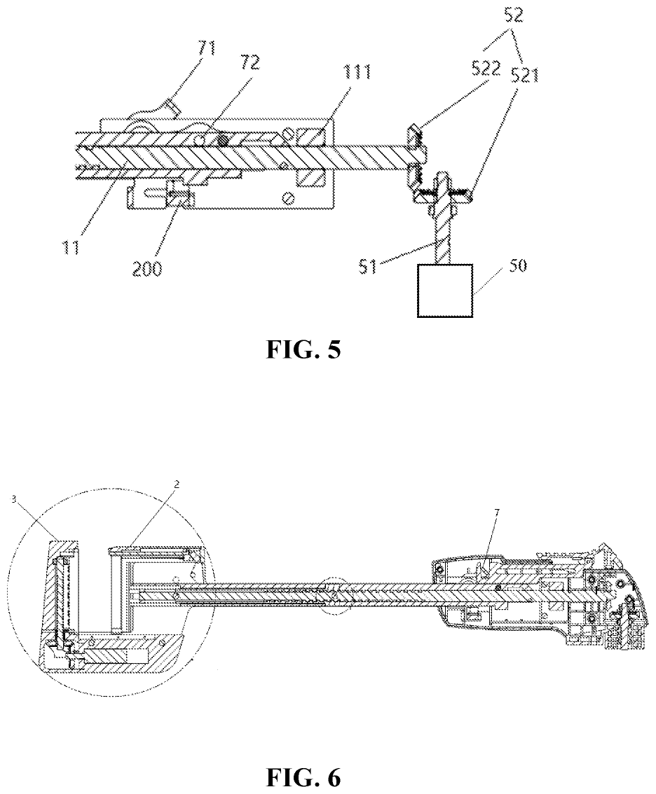

Referring to FIGS. 1 to 5, a surgical instrument according to embodiments of the present disclosure includes: a transmission assembly 1, in which the transmission assembly 1 is provided with a mounting part 101 for mounting a suturing mechanism, the suturing mechanism (i.e. an end effector) includes an anvil assembly 2 and a staple cartridge assembly 3, the staple cartridge assembly 3 includes a staple cartridge 32 for accommodating staples 31, the anvil assembly 2 includes an anvil 21 matched with the staple cartridge 32, and the transmission assembly 1 is connected to the anvil assembly 2 and drives the anvil assembly 2 to move relative to the staple cartridge assembly 3; a staple pushing assembly 4 configured to push the staples 31 to move toward the anvil assembly 2, in which the staple pushing assembly 4 includes a first threaded rod 41 provided on the staple cartridge assembly 3 and a firing nut 42 sleeved on the first threaded rod 41, and an axis direction of the first threaded rod 41 and the firing nut 42 is defined as a first axis direction (a straight line of Arrow A in FIG. 2); a drive assembly 5 connected to the transmission assembly 1 to drive the transmission assembly 1 to move. The surgical instrument of the embodiments of the present disclosure further includes a firing assembly 6 provided on the anvil assembly 2. The firing assembly 6 is connected to the first threaded rod 41 and drives the first threaded rod 41 to rotate, so that the firing nut 42 moves relative to the first threaded rod 41 in the first axis direction.

In the embodiments of the present disclosure, the surgical instrument includes two plate pieces 10 as a main body of the surgical instrument. The transmission assembly 1 is mechanically connected with the plate pieces 10. The suturing mechanism is provided at a distal end of the plate pieces 10 (in a direction indicated by Arrow B in FIG. 2). For example, the distal end of the plate pieces 10 is provided with the mounting part 101. The mounting part 101 is a concave structure. The anvil 21 is provided on a right side of the concave structure. The staple cartridge 32 is mounted on a left side of the concave structure. A staple pushing sheet 33 for accommodating and firing the staples 31 is further provided in the staple cartridge 32. The structure of the staple pushing sheet 33 may refer to common design, which is not detailed here. The anvil assembly 2 further includes a connecting part 22. The connecting part 22 connects the anvil 21 and the mounting part 101. An upper tissue positioning needle 221 is provided in the connecting part 22. An upper tissue positioning hole 341 corresponding to the upper tissue positioning needle 221 is provided on the staple cartridge assembly 3. If the suturing mechanism is closed, the upper tissue positioning needle 221 is inserted into the upper tissue positioning hole 341 to fix the tissue to be sutured. The staple cartridge assembly 3 further includes an upper jaw arm 34 and a lower jaw arm 35. The upper jaw arm 34 is provided at an outer side of the staple cartridge 32 to protect the staple cartridge 32. The upper jaw arm 34 is provided with a slot hole 340 for accommodating the first threaded rod 41 and the firing nut 42. A first bearing 342 for fixing the first threaded rod 41 is provided in the slot hole 340. The lower jaw arm 35 is provided with a hollow chamber 351 for accommodating the firing assembly, and the firing assembly 6 is mechanically fixed in the hollow chamber 351. In the embodiments of the present disclosure, the upper tissue positioning hole 341 is provided on the upper jaw arm 34, and a lower tissue positioning needle 343 is fixed on the upper jaw arm 34. The lower tissue positioning needle 343 is inserted into the anvil assembly 2 if the suturing mechanism is closed.

In the embodiments of the present disclosure, the transmission assembly 1 includes a transmission rod 11. The transmission rod 11 is connected to the drive assembly 5 and rotates under the action of the drive assembly 5. An axis direction of the transmission rod 11 is defined as a second axis direction (a straight line of Arrow B in FIG. 2). The first axis direction is perpendicular to the second axis direction. In the embodiments of the present disclosure, the transmission rod 11 is provided between the two plate pieces 10. The transmission rod 11 is a second threaded rod. A threaded sleeve 12 is sleeved on the second threaded rod 11. The threaded sleeve 12 is preferably provided at a middle portion of the second threaded rod 11. The drive assembly 5 drives the second threaded rod 11 to rotate, then the threaded sleeve 12 moves relative to the second threaded rod 11 in the second axis direction. In the embodiments of the present disclosure, the second threaded rod 11 for example is provided with threads only at the middle portion of the second threaded rod 11. If the suturing mechanism is closed, the threaded sleeve 12 is located at a distal end of the threads of the second threaded rod 11.

In the embodiments of the present disclosure, the firing assembly 6 includes a firing motor 61, a firing input shaft 62 provided on the firing motor 61, and a direction-changing transmission device 63 respectively connected to the firing input shaft 62 and the first threaded rod 41. After the suturing mechanism is closed, the firing motor 61 drives the first threaded rod 41 to rotate by the direction-changing transmission device 63. For example, the direction-changing transmission device 63 includes a first bevel gear 631 sleeved on the firing input shaft 62 and a second bevel gear 632 sleeved on the first threaded rod 41. The first bevel gear 631 and the second bevel gear 632 are engaged with each other to transform a rotation of the firing input shaft 62 with respect to the second axis direction into a rotation of the first threaded rod 41 with respect to the first axis direction. Of course, in other embodiments, the direction-changing transmission device 63 may have other structures, as long as the direction-changing transmission is achieved.

In the embodiments of the present disclosure, the transmission assembly 1 further includes a closure assembly 13 sleeved on the second threaded rod 11. The closure assembly 13 includes a first closure rod 131 and a second closure rod 132. The threaded sleeve 12 is fixedly connected to the first closure rod 131 and the second closure rod 132. The threaded sleeve 12 is provided between the first closure rod 131 and the second closure rod 132. For example, the first closure rod 131 is connected to the connecting part 22 of the anvil assembly 2, and the second closure rod 132 is provided closer to the drive assembly 5 than the first closure rod 131 (i.e. the second closure rod 132 is at a proximal end). The drive assembly 5 drives the second threaded rod 11 to rotate, the threaded sleeve 12 moves relative to the second threaded rod 11 in the second axis direction, thereby driving the first closure rod 131 and the second closure rod 132 to move in the second axis direction. The first closure rod 131 is connected to the anvil assembly 2, so that during the threaded sleeve 12 moves toward the distal end of the second threaded rod 11, the first closure rod 131 pushes the anvil assembly 2 to move toward the staple cartridge assembly 3 to close the suturing mechanism. The first closure rod 131 and the second closure rod 132 are integral with each other, or are provided separately and then connected together. Preferably, the first closure rod 131 and the second closure rod 132 are integral with each other, and are provided with a slot hole (not shown) for receiving the threaded sleeve 12.

In the embodiments of the present disclosure, a protruding rod 1311 is provided on the first closure rod 131. The protruding rod 1311 abuts against a proximal end of the upper tissue positioning needle 221. During the first closure rod 131 drives the anvil assembly 2 to move toward the staple cartridge assembly 3, the protruding rod 1311 drives the upper tissue positioning needle 221 to move toward the upper tissue positioning hole 341 and insert into the upper tissue positioning hole 341, so that the tissue to be sutured is positioned by the upper tissue positioning needle 221 so as to facilitate subsequent suturing and cutting.

The surgical instrument of the embodiments of the present disclosure further includes a safety switch 7 serving as a stopper. The safety switch 7 is provided on the closure assembly 13. For example, the safety switch 7 includes a switching knob 71 and a switching shaft 73. The switching shaft 73 is connected to the switching knob 71. The second closure rod 132 is provided with a through hole 72 adapted to the switching shaft 73. After the suturing mechanism is closed, the switching knob 71 is toggled to insert the switching shaft 73 into the through hole 72 to lock the closure assembly 13. The safety switch 7 may refer to common design, which is not detailed here.

The surgical instrument of the present disclosure further includes a handle 100. The transmission rod 11 is fixed to the handle 100 by a second bearing 111. The second closure rod 132 and the drive assembly 5 are fixed to the handle 100. A manner of fixing may refer to common manner, such as a thread connection manner, a bonding manner, or a snap connection manner, which is not limited herein. The drive assembly 5 includes a drive motor 50, an input shaft 51 connected to the drive motor 50, and a transmission member 52 respectively connected to the input shaft 51 and the transmission rod 11. The transmission member 52 includes a first bevel gear 521 sleeved on the input shaft 51 and a second bevel gear 522 sleeved on the transmission rod 11. The first bevel gear 521 is engaged with the second bevel gear 522 so as to transform a rotation of the input shaft 51 with respect to the first axis direction into a rotation of the transmission rod 11 with respect to the second axis direction.

In the embodiments of the present disclosure, the surgical instrument further includes an induction switch 200 for sensing a position of the staple cartridge assembly 3. The induction switch 200 is fixed in the handle 100; and a manner of fixing may refer to common design, such as the thread connection manner, the bonding manner, or the snap connection manner, which is not limited herein. The induction switch 200 is a Hall switch or a photoelectric switch. After the suturing mechanism is closed and the closure assembly 13 is stationary, the staple cartridge assembly 3 at this time is provided at a position that the staple cartridge 32 and the anvil 21 are closed together, and the position of the staple cartridge assembly 2 is acquired by the induction switch 200.

In the embodiments of the present disclosure, a plugging hole for external power (not shown), a step indicator (not shown), and an operation button (not shown) are also provided on the handle 100. The plugging hole for example is connected to a power line of a medical power supply. Of course, the instrument may be powered by a built-in power supply, such as a battery.

Preferably, the surgical instrument of the embodiments of the present disclosure is a linear surgical stapler. The suturing mechanism is mounted on the mounting part 101. The suturing mechanism includes the anvil assembly 2 and the staple cartridge assembly 3 as described above.

The linear surgical stapler operates as follows.

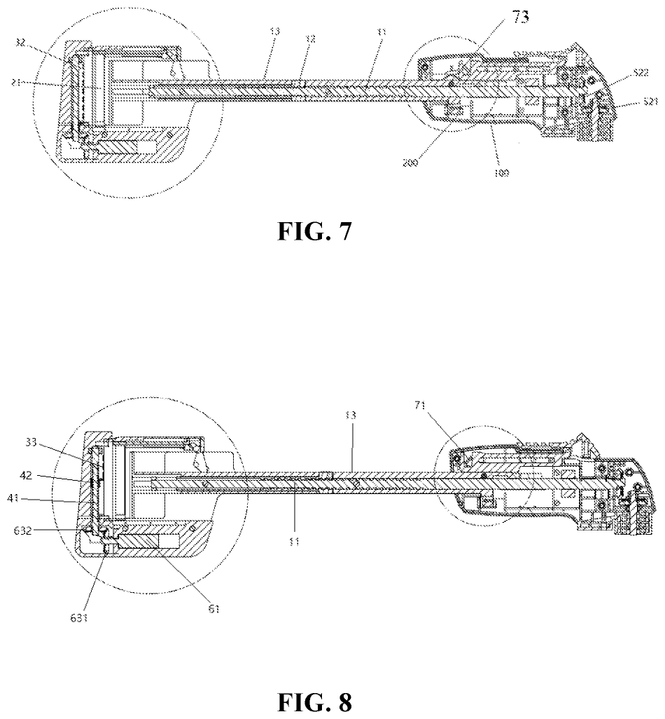

Referring to FIG. 6, in an initial state, the anvil assembly 2 is spaced from the staple cartridge assembly 3, and at this time, the safety switch 7 is in an “open” state at this time. Referring to FIG. 7, the operating button on the handle 100 is pressed, and the drive motor drives the input shaft 51 to rotate; due to the engagement between the first bevel gear 521 and the second bevel gear 522, the second threaded rod 11 is driven to rotate, the threaded sleeve 12 sleeved on the second threaded rod 11 is driven to push the closure assembly 13 to move toward the distal end of the second threaded rod 11 until the target tissue is clamped by the anvil assembly 2 and the staple cartridge assembly 3, the suturing mechanism is in the closed state (i.e. a state in which the target tissue is clamped by the anvil assembly 2 and the staple cartridge assembly 3), and the closure assembly 13 is stationary; at this time, the staple cartridge 32 is at the position that the staple cartridge 32 and the anvil 21 are closed together, the position of the staple cartridge 32 is acquired by the induction switch 200 (such as the photoelectric switch). Referring to FIG. 8, after the suturing mechanism is in the closed state, the switching knob 71 is toggled, and the safety switch 7 is in a “locked” state; at this time, the closure assembly 13 is locked; then, the firing motor 61 is driven to rotate, the first bevel gear 631 and the second bevel gear 632 are engaged with each other to drive the first threaded rod 41 to rotate, so that the first threaded rod 41 drives the firing nut 42 to move upward in the first axis direction, thereby pushing the staple pushing sheet 33 to sequentially fire the staples. The firing nut 42 automatically returns to an initial position after the firing is completed. After the firing of the staples is completed, the firing nut 42 automatically returns to the initial position. After the firing of the staples is completed, the safety switch is turned back to the “open” state, the drive motor rotate reversely, the second threaded rod 11 rotate reversely to drive the closure assembly 13 to move back so that a distance between the staple cartridge assembly 3 and anvil assembly 2 restores to the maximum (which may be controlled by the number of turns of the drive motor), which is consistent with the initial state.

In summary, the surgical instrument and the linear surgical stapler of the present disclosure are provided with the staple pushing assembly and the firing assembly. If the suturing mechanism is closed, the firing assembly drives the first threaded rod of the staple pushing assembly to rotate so as to drive the firing nut sleeved on the first threaded rod to move relative to the first threaded rod in the first axis direction, so that the staples in the staple cartridge are fired sequentially. Therefore, there is no problem such as large and small heads, dovetail and the like. In addition, the surgical instrument and the linear surgical stapler fire the staples sequentially, which can reduce the firing force to avoid the deformation of the anvil, and have a better insertion effect of the staples into the tissue, a better suturing effect and surgical safety.

The technical features of the foregoing embodiments may be combined arbitrarily. In order to make the description concise, all possible combinations of the various technical features in the foregoing embodiments are not described. However, as long as there is no conflict in the combination of these technical features, all combinations should be considered as within the scope of the disclosure.

The foregoing embodiments merely are some of the embodiments of the present disclosure and the descriptions of the foregoing embodiments are specific and detailed, but the foregoing embodiments should not be understood as limiting of the scope of the disclosure. It should be pointed out that a person of ordinary skill in the art may make modifications and improvements without departing from the concept of the present disclosure, and all of these modifications and improvements belong to the protection scope of the present disclosure. Therefore, the protection scope of the present disclosure should be defined by the appended claims.

1. A surgical instrument, comprising:
a transmission assembly, wherein the transmission assembly is provided with a mounting part for mounting a suturing mechanism, the suturing mechanism comprises an anvil assembly and a staple cartridge assembly, the staple cartridge assembly comprises a staple cartridge for accommodating staples, the anvil assembly comprises an anvil matched with the staple cartridge, and the transmission assembly is connected to the anvil assembly and drives the anvil assembly to move relative to the staple cartridge assembly;
a staple pushing assembly configured for pushing the staples to move toward the anvil assembly, wherein the staple pushing assembly comprises a first threaded rod provided on the staple cartridge assembly and a firing nut sleeved on the first threaded rod, an axis direction of the first threaded rod and the firing nut is defined as a first axis direction; and
a drive assembly, connected to the transmission assembly to drive the transmission assembly to move; wherein
the surgical instrument further comprises a firing assembly, the firing assembly is connected to the first threaded rod and drives the first threaded rod to rotate, so that the firing nut moves relative to the first threaded rod in the first axis direction.

2. The surgical instrument according to claim 1, wherein
the firing assembly comprises a firing motor, a firing input shaft provided on the firing motor, and a direction-changing transmission device respectively connected to the firing input shaft and the first threaded rod; and
if the suturing mechanism is closed, the firing motor drives the first threaded rod to rotate by the direction-changing transmission device.

3. The surgical instrument according to claim 2, wherein the direction-changing transmission device comprises a first bevel gear sleeved on the firing input shaft and a second bevel gear sleeved on the first threaded rod, and the first bevel gear and the second bevel gear are engaged with each other.

4. The surgical instrument according to claim 1, wherein the transmission assembly comprises a transmission rod, the transmission rod is connected to the drive assembly and rotates under an action of the drive assembly, an axis direction of the transmission rod is defined as a second axis direction, and the first axis direction is perpendicular to the second axis direction.

5. The surgical instrument according to claim 4, wherein
the transmission rod is a second threaded rod, and a threaded sleeve is sleeved on the second threaded rod;
the drive assembly drives the second threaded rod to rotate, so that the threaded sleeve moves relative to the second threaded rod in the second axis direction.

6. The surgical instrument according to claim 5, wherein
the transmission assembly further comprises a closure assembly sleeved on the second threaded rod, the closure assembly comprises a first closure rod and a second closure rod, the threaded sleeve is fixedly connected to the first closure rod and the second closure rod, the threaded sleeve is provided between the first closure rod and the second closure rod
the first closure rod is connected to the anvil assembly, the second closure rod is provided closer to the drive assembly than the first closure rod;
if the drive assembly drives the second threaded rod to rotate, the threaded sleeve moves; relative to the second threaded rod in the second axis direction, thereby driving the first closure rod and the second closure rod to move in the second axis direction.

7. The surgical instrument according to claim 6, wherein
the surgical instrument further comprises a safety switch serving as a stopper, the safety switch is provided on the closure assembly, the safety switch comprises a switching knob and a switching shaft, the switching shaft is connected to the switching knob, and the second closure rod is provided with a through hole adapted to the switching shaft; and
if the suturing mechanism is closed, the switching knob is toggled, the switching shaft is inserted into the through hole to lock the closure assembly.

8. The surgical instrument according to claim 1, wherein
the further comprises an upper jaw arm and a lower jaw arm, the upper jaw arm is provided at an outer side of the staple cartridge, the upper jaw arm is provided with a slot hole for accommodating the first threaded rod and the firing nut;
the lower jaw arm is provided with a hollow chamber for accommodating the firing assembly, and the firing assembly is mechanically fixed in the hollow chamber.

9. The surgical instrument according to claim 4, wherein the surgical instrument further comprises a handle, the transmission rod is fixed to the handle, the drive assembly is provided in the handle, and the drive assembly comprises a drive motor, an input shaft connected to the drive motor, and a transmission member respectively connected to the input shaft and the transmission rod.

10. The surgical instrument according to claim 9, wherein the surgical instrument further comprises an induction switch for sensing a position of the staple cartridge assembly, and the induction switch is provided in the handle.

11. A linear surgical stapler, comprising a surgical instrument according to claim 1 and the suturing mechanism, wherein the suturing mechanism is mounted on the mounting part.

12. The surgical instrument according to claim 1, wherein
the anvil assembly further includes a connecting part, the connecting part connects the anvil and the mounting part;
an upper tissue positioning needle is provided in the connecting part, an upper tissue positioning hole corresponding to the upper tissue positioning needle is provided on the staple cartridge assembly;
a protruding rod is provided on the transmission assembly, and during transmission assembly drives the anvil assembly to move toward the staple cartridge assembly, the protruding rod drives the upper tissue positioning needle to move toward the upper tissue positioning hole and insert into the upper tissue positioning hole.

13. The surgical instrument according to claim 4, wherein the surgical instrument includes two plate pieces as a main body of the surgical instrument, the suturing mechanism is provided at a distal end of the plate pieces, and the transmission rod is provided between the two plate pieces.

14. The surgical instrument according to claim 5, wherein
the second threaded rod is provided with threads only at a middle portion of the second threaded rod; and
if the suturing mechanism is closed, the threaded sleeve is located at a distal end of the threads of the second threaded rod.

15. The surgical instrument according to claim 8, wherein a lower tissue positioning needle is fixed on the upper jaw arm, and the lower tissue positioning needle is inserted into the anvil assembly if the suturing mechanism is closed.

16. The surgical instrument according to claim 9, wherein the transmission member includes a first bevel gear sleeved on the input shaft and a second bevel gear sleeved on the transmission rod, the first bevel gear is engaged with the second bevel gear so as to transform a rotation of the input shaft with respect to the first axis direction into a rotation of the transmission rod with respect to the second axis direction.
